(19) United States
(12) Patent Application Publication
(10) Pub. No.: US 2008/0038362 A1
(43) Pub. Date: Feb. 14, 2008

(54) Composition For An Enteric Coating Of Natural Product Containing Lectin

(76) Inventors: Won-Bong Park, Seoul (KR); Su-Yun Lyu, Souel (KR)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(21) Appl. No.: 10/559,988

(22) PCT Filed: Mar. 19, 2004
(86) PCT No.: PCT/KR04/00606

§ 371 Date: Dec. 9, 2005

(30) Foreign Application Priority Data
Jun. 12, 2003 (KR) 10-2003-0037994

(51) Int. Cl. A61K 9/50

(52) U.S. Cl. 424/491

(57) ABSTRACT

This invention is accomplished by producing an effective composition for enteric coated medicine of mistletoe extract C) containing lectin and producing an effective composition for enteric coated microcapsule which has lectin as a major ingredient. The problem with oral administration due to unstability in the digestive system was solved and it enhanced drug efficiency, medical cure effectiveness contributing to medical industry.

### TECHNICAL FIELD

The present invention relates to a composition for enteric coating of mistletoe extract containing lectin. Also, the invention relates to a composition for enteric-coated microcapsules which contains lectin as a major ingredient.

### BACKGROUND ART

Lectin can be obtained from various plants such as mistletoe, Cornus, officinalis, an evening primrose, a kidney bean, beans, Liriope muscari, peony, sangryuk, papaya, a ground cherry, todangui, ivy, goosefoot, guallugun, shitake mushroom, Pinellia ternata, the boxwood tree, an acacia and also various natural substances from the sea such as starfish and mudfish. At present, lots of lectins are in product on a commercial scale and concanavalin A (con A) or lectin from kidney beans (PHA), ricin, abrin etc. are used in many researches. Lectin is a glycoprotein or a glucose binding protein that has more than two glucose binding sites. It agglutinates erythrocyte and other blood cells and precipitates carbohydrate compounds. Various biochemical and immunological properties of lectin lead to using it for the therapeutics, diagnosis and research methods for life science (Chung et al., Arch Pharm Res vol. 40(4): 387-393,1996).

One of the most important biochemical and immunological properties of lectin is a selective agglutination of the tumor cells, specificity against human blood cells, mitogenic activity which stimulates differentiation of lymphocytes in stoppage, etc. Lectins which has from 2 to 6 glucose binding sites, has a B-chain (binding chain) is capable of binding to cell surface receptor (specific carbohydrates) and thereby permits entry into the cell of the A-chain (active chain) which inactivates the ribosomes resulting in inhibition of protein synthesis. Binding the cell surface carbohydrate with B-chains is similar to antigen-antibody reaction and this binding specificity plays an important direct role for regulating the immune system or anti-cancer activity. It has reported that cancerous lymphocytes are different from the normal lymphocytes having agglutinative activity by lectin and this fact became a background for studying alterations of the cancerous cell membranes using lectin. When the lectin is given to the lymphocyte, the previous differentiated lymphocytes divide and proliferate to lymphoblasts.

One of the immunological features of the physiological activities of lectin is a mitogenic activity towards the lymphocyte. According to the types of lectin, it stimulates T or B cells. The mechanism for the T cell is that lectin stimulates the macrophage to secrete interleukin-1 (IL-1) and this activates the helper T cell to secrete IL-2 for the proliferation of the T cell. The mechanism for the B cell is that lectin acts directly or the interferon-γ(IFN-γ), IL-4, IL-5 and IL-6 secreted from helper T cell to proliferate B cells.

Secondly, there is an anti-cancer activity. As a single cell population antibody activated from cancer cell antigen's growth is given with lectin, growth of the cancer cell is inhibited from inhibition of proteins synthesis (Vieta et al., Science 219: 644,1983), macrophage or poly-nucleated leucocytes lyses the cancer cells with existence of lectin (Ohkuma et al., Cancer Res. 45, 4397,1985). And the mechanism of anti cancer activity of the cytokines (IFN-γ, IL-2, TNF-α) secreted by the T cell or macrophage activated with lectin has been reported (Tamura et al., FEBS Lett. 175:325-328,1984).

In addition, there is an insulinomimetic activity by lectin. Binding with the insulin receptor of adipocyte to promote glucose transport and metabolism, promoting lipogenesis and pyruvate dehydrogenase activity, promoting glycogen synthesis and Mg-ATPase activity, inhibiting lipolysis and adenylcyclase activity of lectin is reported (Suya et al., J. Biochem. 92:1251-1257,1982).

As mentioned above, nevertheless the lectin included in natural sources has various physiological activities, we lose the virtue of the lectin due to the digestion of the lectin protein to amino acids in the small intestines to be absorbed into the blood circulation system (Pusztai A. Lectins. Toxicants in plant origin, Vol III, 1987).

Mistletoe (Viscum album), one of the lectin including plant was used in cancer treatment for a long time and it has few side effects having excellent anti cancer activity on the other side (Hajto et al., Cancer Research 50:3322-3326,1990. Jassen et al., Drug Research 43(11): 1221-1227,1993, Am. Soc. Biochem. and Molc. Biol. 267(33): 23722-23727,1992). This plant is reported that it is effective for killing cancer cells directly and complex working of immunity activation, stimulating humoral and cell-mediated immunity, activating macrophage and natural killer (NK) cell to inhibit the cancer cell and increasing the survival rate of the cancer patients (Jassen et al., Drug Research 43(11): 1221-1227, 1993).

This plant contains lectin with M.W. 60 kDa, viscotoxin, polysaccharides, other active factors and the most effective component is lectin (Bussing et al., Cancer Lett. 92: 199-205,1995, Cancer Lett. 99: 59-72,1996., Jung et al., Cancer Letters 51:103-108,1990). The anti cancer effects acquired by the anti cancer drugs induce death or inhibit abnormal growth of the cancer cell. Cytoxicity against tumor cells and leucocytes by mistletoe is the result from the induction of apoptosis and it was reported that only lectin could induce this process (Bussing et al., Cancer Lett. 94:199-205,1995, Cancer Lett. 99: 59˜72,1996).

Korean mistletoe (Viscum album, L. coloratum), a variety of European mistletoe, has also been reported to be superior to European mistletoe in its efficiency respect but the research on it is insufficient (Park et al., Arch Pharm Res vol. 38(4): 418-424, 1994, Arch Pharm Res vol. 39(1): 24-30, 1995, Arch. Pharm. Res. 20(4):306˜312, 1997, Arch. Pharm. Res. 21(40):429˜435, 1998, Foods and biotechnology 8(4): 232˜237, 1999).

The present inventors extracted lectin from Korean mistletoe and it came out to have similar cytotoxicity effects with European mistletoes, losing this activity without lectin in the mistletoe extract (Park et al., Food Sci. and Biotechnol. 8: 391˜396, 1999., Foods and Biotechnol. 8(4): 232˜237, 1999). Also, genes and amino acid sequences were analyzed and through column chromatography they extracted pure lectin to find out that it increased immunity, anticancer effects and they applied for a patent (Korean Patent Application No. 2000-83383) for ELLA (enzyme linked lectin assay) which is used when determining the concentration of mistletoe lectin. Moreover, the water extraction of the mistletoe has few side effects and is effective for inhibition at metastasis of all kinds of cancer including skin cancer. Furthermore, they developed lectin fortified mistletoe extract and an anti cancer composition which contains this using cytotoxicity caused by apoptosis, antiangiogenesis, inhibiting telomerase activity by the lectin. And they developed and applied for a patent (Korean Patent Application No. 2001-0061118) on a medicine that can be medicated directly on the affected part of skin or oral cancer.

Nevertheless the main factor for the anticancer effect is lectin, present mistletoe medicines produced are complex compound of water extracts and it is reported that this is more effective than single lectin. This is because other compounds like viscotoxin, alkaloid and more have synergistic effects together. Particularly, it has been reported that viscotoxin with 5 kDa has anti cancer effects too (Schaller et al., Phytotherapy Res. 10: 473˜477). And also the fact that lectin in complex compound is more stable than the single lectin can be another reason.

Like this the mistletoe has an effective anti cancer activity without side effects. So, if delivery via oral route is possible, it can be used extensively in treatment and prevention of cancer. And it is reported that if lectin is orally delivered from 10 to 1000 times more dosage than hypodermical injection (0.05 mg˜3 mg/kg body weight), it strongly binds with Peyer's patch M-cell, inhibits the growth of tumor cell, promotes secretion of cytokine TNF-α, IL-1β and more (Pusztai et al., J. Nutr. Biochem. 9: 31˜36, 1998). But taking high dosage of lectin considering the breakdown in the digestive system is uneconomical and other compounds not being digested in the intestine can make side effects. That is to say, even though mistletoe extracts can be used widely as anti cancer treatment and prevention, it has only been developed as an injection not using it broadly.

The oral administration is mostly used in making medicine and for the effect of it, the medicine must pass the barriers and reach the blood circulation. Orally delivered medicine goes down the esophagus, intestine and be dissolved and release drugs. Human digestive system is several meters long taking lots of time to pass through it. While passing through, the pH in digestive tract is changed from acidic to neutral and to weak alkali, making the medicine to be exposed to various enzymes and the inside of the intestine. Meanwhile, the medicine is dissolved to molecules and is absorbed through the digestive mucous membranes. Most of the medicines go to the liver through the stomach mesentric vein and provide first time passed effect. Therefore, because of the unstability in the intestine or low permeability through the mucous membrane, it's hard to develop oral medicine. In particular, while oral delivery, protein or peptides like insulin, interferon and glycoproteins like lectin are digested to amino acids or small peptides. The peptides in the small intestine epithelium, quickly dissolved to amino acids by the aminopeptidase gets absorbed into blood circulation. In consequence, using oral administration the effect of medicine declines.

In this case, we can enhance the stability of the medicine in the intestine to improve absorption. First of all, the medicine must not get dissolved by the gastric acid. So, enteric coated medicines were developed to avoid acidic or pepsin mediated decomposition by encapsulating medicines. Enteric coated medicines (tablet or granule) don't melt at acidic or neutral pH but when it comes to the intestine, it melts on account of alkaline intestinal juices so that it can release drugs selectively. Also, to solve with decomposition problem, a system using proteinase inhibitors (e.g. aprotinin, soybean trypsin inhibitor, bestatin etc) was attempted (Drug Delivery Rev., 4: 171, 1990).

Generally, the low molecular compounds (less than MW 5000) existing in intercellular space can pass through a capillary vessel to reach the circulation. On the contrary, high molecular compounds, particles and substances which forms a big chylomicron due to its lipophilic property is too big to pass the capillary that they pass through the mucous membranes to reach lymphatic vessels and to the body. In the intestinal mucous membrane, there are few Peyer's patch which contains M (microfold)-cells and lymphatic systems. But, the lymph flow is 1/200˜ 1/500 times smaller than the blood, so it's hard for the medicine to get absorbed into the lymphatic vessels. Special compounds like Vitamin A, lipids like cholesterol, vitamin B₁₂ and its derivatives, lectin containing compounds, liposomes and ultra microparticles (less than 10 μm diameter) and others don't pass the M-cell to the lymphatic vessel but pass through into the blood vessel directly. Therefore, if we allow these compounds to have the intestinal wall permeability, they can be used as lymphatic vessel carriers.

If medicine can be absorbed into the lymphatic system, it will pass through the intestinal lymph vessel and thorax lymph vessel to the blood vessel avoiding passing the liver the first time. Therefore, if we give lectin the permeability through the mucous membrane it will pass the M-cell in the Peyer's patch and pass through the lymphatic vessel and directly to the blood vessel without passing the liver after all being used as lymphatic vessel carriers. Particularly, the lymphatic system is a passage for cancer metastasis or bacterial infection so lymph nodes can be the cause of disease. Consequently, for selectively sending anti cancer treatments or antibiotics to the lymph nodes for treatment or diagnosis, lectin can be used.

Medicines are made convenient to apply and processed to be most suitable to be effective. When it is taken the drug is released then through absorption, distribution, metabolism, excretion it acts as a treatment. To use it more stably and effectively we need technique to control the action of the medicine. Drug Delivery System (DDS) is for reducing side effects and maximizing the effects to deliver needed amount of medicine. Controlled release system has capsules for oral administration, matrix style, microcapsules for oral administration or injection, microsphere, microparticle, nanoparticle, liposomes and implants and others.

Microcapsules can be defined as many words according to the shape and size processed. Namely, microcapsule is a ball-shaped particle which has solid or liquid drugs placed in the middle nucleus and microsphere is a multinucleated microcapsule which contains distributed solid or liquid drugs. Moreover, microparticles includes both microcapsule and microsphere and it's a particle type drug carrier which carries high molecular matrix or lipid particles. Among these, some has diameter less than 1 μm which is called the nanosphere (or nanoparticle). Hereinafter, unless these words are used differently, they mean as mentioned above.

Liposomes have similar structure to the membrane, so it is used for drug delivery system and its function. Liposomes comprises phospholipids dissolved in the body with no cytotoxicity and due to its amphiphilic property, it captures both hydrophilic and hydrophobic drugs. Also, it captures drugs into the inside to inhibit drug inactivation, enhance utilization of the peptide-kind of drug, administer any kind of drug to almost any kind of delivery channels, targets specific tissue to promote medical cure. Liposomes can be classified into multilamellar vesicles (MLV) which has bilayers in many folds, uniamellar vesicles which is composed of a single layer according to the structure, and this uniamellar vesicles can be classified into small uniamellar vesicles (SUV), large uniamellar vesicles (LUV) and others according to the size. The SUV is 20˜50 nm and LUV is 100˜1000 nm sized.

Solid-lipid nanoparticles (SLN) is nano-particle oral delivery system produced for using hydrophobic drugs. Compared to high molecular particles or liposomes, it enhances chemical stability of the captured drugs, controls the release of the drug and inhibits the aggregation between the particles.

Microcapsulation is a technique which enclose minute solid or liquid particles with various coating materials or process the mixed form in it from the size 0.1 μm to hundreds μm. That is to say, microcapsule is a particle which is made by special reactions or manipulation to capsulate drugs, coating materials, additives and solvent agents. Therefore, we can capsulate solid, liquid, gas to a microscopic view.

During microcapsulation of protein, proteins are exposed to excess stress. Proteins have high molecular weights and because its activity and physical property depends on the three-dimensional structure, it's apt to denature more than other chemically synthesized drugs. Therefore, microcapsule processes of protein drugs must be blocked from excess heat, shear stress, extreme pH change, organic solvent, freeze, dryness. Besides, during the storing, the microcapsulated protein can be hydrated and this easily leads to aggregation, denaturalization and inactivation. In consequence, biodegradable polymer must be used and denaturalization must be blocked and also the encapsulating efficiency must be high enough when making the protein or peptide containing microcapsule. Moreover, simple process, the minimum use of organic solvents and mass production must be practicable.

Microcapsulation technique is various that core substances and final particle size depends on the process. Substances used in the process are various synthetic and natural high molecular compounds. Also high molecular substances which decomposes in the body like albumin, gelatin, collagen, fibrinogen, alginate, starch, poly amino acid, polylactides (PLA), polyglycolides (PGA), poly β-hydroxy butyric acid (PHB), polycaprolactone, polyanhydrides, polyorthoesters and PLGA which is a mixture of these are used. Moreover, there is airsuspension, phase-separation, airspraying, orifice/centrifugal method, supercritical fluid technic, pancoating, solvent-evaporation, spray drying and coagulation, surface polymerization, melt cooling.

Multiple emulsion solvent evaporation method is dissolving high molecule solvent in volatile organic solvent (OS) which the drug is dissolved in distilled water or buffer solution (inner water phase: IWP) without mixing with water. After emulsifying the IWP in organic solvent and making primary emulsion (W/O), pour this to emulsifier containing outer water phase (OWP) and agitate to make secondary emulsion (W/O/W). Agitating this multiple emulsion continuously and evaporating the organic solvent to induce precipitation of the high molecule, and then the drug-loaded microparticle is formed. The emulsifier in the OWT has an important role in formation of the spherical microparticle. This is for preventing coagulation between the particles while the organic solvents evaporate. Generally, polyvinylalcohol (PVA) is used but polyvinylpirrolidone, alginates, methylcellulose, gelatine can be used also as an emulsifier. Organic solvents can be removed under normal pressure or decompression state.

Most popular way for drug delivery is oral administration. However, it's difficult because of the unstability while being digested in the intestine or the low permeability for the mucous membrane. For example, insulin, interferon and other proteins or peptides, glycoproteins like lectin get digested to amino acids or small peptides. So to speak, medicines for oral delivery dissolves and releases its drugs while passing the digestive system because the pH changes throughout the digestive system, acidic to neutral and to basic and also contacts with various enzymes. Peptide which has entered the epithelium cells of the small intestine, decomposes into amino acids with the actions of aminopeptidase and gets absorbed into blood circulation by amino acid carriers. Therefore, the proteins lose their function.

Consequently, the natural substances of the lectin delivered orally couldn't be used widely in spite of the use in medical cure. In this case, we can improve the absorption by increasing the stability of drugs in the digestive system. In order to improve this, various enteric coated medicine were developed for preventing the decomposition at the stomach and avoiding acid and pepsin-related proteins. Enteric coated medicines (tablet or granules) withstand the low pH in the stomach and then melts in the intestine due to high pH to release drug in a selective way. Enteric coating can be applied to not only tablets but also granules and the thickness of the covering can be regulated. Shellac, hydroxypropylmethyl cellulose phthalate, polyvinylacetate phthalate, cellulose acetate phthalate, Zein, Eudragit L100, S100, alginate, gelatin, starch and others are used in single or mixed form for enteric coating. There are many ways for coating substances like using the fan coating apparatus, fluid bed coater, spraying to make a coat around, powder coating technique using static electricity, dry coating, hot-melt coating and others and they are used singular or together.

In consequence, this invention provides ways to produce tablets or granules containing mistletoe extracts and lectin which is difficult for oral administration and produce enteric coated medicines by coating the tablets or granules using coating tools and plasiticizers in low temperature. Furthermore, this invention provides ways to produce double enteric microcapsule after producing the lectin containing microcapsule.

### BRIEF DESCRIPTION OF THE DRAWINGS

FIG. 1 is a picture of the surface structure from the alginic acid-salt double microcapsule.

FIG. 2 shows elution rate of mistletoe extracts containing lectin from an alginic acid double microcapsule.

### DISCLOSURE

### Technical Problem

An object of this invention is to produce an effective composition for enteric coated medicine of mistletoe extract containing lectin and an effective composition for enteric coated microcapsule which has lectin as a main ingredient.

### Technical Solution

This invention is accomplished by producing an effective composition for enteric coated medicine of mistletoe extract containing lectin and producing an effective composition for enteric coated microcapsule which has lectin as a main ingredient.

The excipient for enteric coated medicine of mistletoe extract containing lectin, comprises mannitol 115 g as a excipient, Avicel PH 101 18 g, calcium-phosophate dibasic 17 g, hydroxypropylmethylcellulose 20 g as a binder solution, water 100 mL, ethanol 100 mL, Zein-DP 25 g as a coating solution, Shellac 35 g, 80% ethanol 180 mL.

Also, enteric coated microcapsule which has lectin as a main ingredient comprises PLGA/CH₂Cl₂ 4 mL, 1% polyvinylalcohol 50 mL, Span80 2 mL as a surface active agent, edible oil 48 mL, 1˜4% sodium alginate solution 8 mL, 0.02˜0.2 M CaCl₂ solution 60 mL.

The enteric coating of mistletoe extract containing lectin was produced by using special coating materials singularly or in a complex like shellac, hydroxypropylmethylcellulosephthalate (HPMCP, Pharmacoat 606, Pharmacoat 645), polyvinylacetate phthalate, cellulose acetate phthalate, Zein, Eudragit L100, Eudrgit S100, alginate, gelatin, starch and others. A process for coating is various using singularly or in a complex for instance, fan coating apparatus, fluid bed coater, spraying to coat, dry-coating, hot-melt coating and others.

The mistletoe extract containing lectin is desirable to have 1˜95 weight percentage (%) in the whole coated particle. The coating solution is produced by dissolving coating reagent and plasticizer in an appropriate solvent. Coating reagent in this process are used singularly or in a complex using methacrylic acid copolymer Eudragit (registered a trademark) E-100, Eudragit L3D (Rohm & Hass company, Germany), corn protein extract (Zein-DP) and artificially processed goods using these to produce for instance, sodium alginate, alginic acid, shellac, carboxyvinylpolymer (carbomer (registered a trademark)), hydroxypropylmethylcellulose phthalate, hydroxypropylmethylcellulose acetate succinate, hydroxyproprylmethyl acetate succinate, carboxymethylcellulose, cellulose acetate phthalate, hydroxypropylcellulose, ethylcellulose, methylcellulose, polyvinylacetate phthalate, soy protein, wheat protein, processed goods using soy or wheat proteins, chitin, chitinic acid, processed goods using chitin or chitinic acids, gelatin, Carrageenan, pectin, Guar gum, Locust bean gum, Xanthan gum, Gellan gum, Arabic gum, Kollicoat MAE 30 DP (BASF company), medium chain triglycerides which has 6˜12 carbons. The coating reagent is desirable to have 1˜50 weight percent in the whole coated particle.

The plasticizer in this invention is used singularly or in a complex using, polyethylene glycol, glycerin fatty acid ester, sorbitan fatty acid ester, propylene glycol, glycerin, citric acid triethyl, triacetin, cetyl alcohol, stearoyl alcohol and the plasticizer is desirable to have 0.5˜50 weight percentage (%). If the above stated coating reagents and plasticizers are used out of the range, the solubility of the coated particle decreases leading to unstable coating and late medicine effects.

The solvent in this invention is used singularly or in a complex using, water, ethanol, alcohol (methanol, isopropylalcohol), acetone, acetonitril, methylenechloride, ether, nucleotides, chloroform, 1,4-dioxane, tetrahydrofuran, dimethyl sulfoxide, ethylacetate, methylacetate.

The excipient in this invention is starch, lactose, non-crystalline cellulose, light anhydrous silicic acid, calcium phosphate dibasic, Ac-di-sol, polyvinylpyrolidon (PVP) K-30 and others, and the excipient is desirable to have 0.5˜90 weight percentage (%) in whole coating particle.

To produce an enteric coated medicine, fluid bed erector, high speed mixer, cylinder type granule former is needed. The apparatus needed is fluid bed coater, CF-granule former and particularly in this invention the fluid bed coater Granule-40 (Freund Co. Japan) which is similar to those mentioned above were used and also other similar apparatus can be used.

The temperature of the apparatus needed for producing enteric coated granule is 35˜70° C. Also, for the inside of the apparatus in every processing step, the temperature is desired to be 25˜60° C. because under 25° C., the hygroscopic granules aggregate together and over 60° C., the granules yet put in the process or under formation falls to pieces. Now, the normal temperature depends on seasons so it must be controlled for instance, during the rainy season or winter temperature must be set slightly higher and during summer it must be slightly lower for coating.

In addition, the microcapsulation of the lectin protein uses double emulsion method. And high molecular substances are decomposable for example, albumin, gelatin, collagen, fibrinogen, polylactide (PLA), polylactide-co-glycolides (PLGA)-like hydroxy acid, polylactide-co-glycolides (PLGA), PEG, poly β-hydroxy butyric acid (PHB), polycaprolactone, polyanhydrides, polyorthoesters, polylactide-co-glycolides (PLGA), polyuretan, polybutylates, polyvalerylate, polylactide-co-caprolactone and its derivatives, copolymers or a mixture can be used. The word ‘derivatives’ means transformable high molecular compound by chemical groups such as alkyl or alkylenes. In general, both enzymatic or nonenzymatic hydrolysis occurs in biodegradable high molecular compounds causing surface or bulk erosion.

Microcapsulation of lectin using double-emulsification solvent evaporation procedure is described below.

Adding lectin solution into high molecular compound solution (e.g. PLGA/DCM) to make primary emulsion solution, putting this slowly into emulsifier (e.g. 1% PVA solution), and then secondary emulsion solution was made. After this, stir to solidify the high molecular compounds and centrifuge to collect particles, and wash three times with water to gain microcapsules.

Lectin-releasing controller may include pharmacologically acceptable excipients, carriers or additives. Also, it can be produced into inhalation-administrated type, oral administrated type, injection, sclerite-absorbed type. Furthermore, taking advantage of the formation technology as in the past, lectin-releasing controller can be produced by using the microcapsule (e.g. crystal/PLGA).

And also, through the reverse phase evaporation (REV) namely, giving supersonics to the solution which is lectin solution dissolved in phophatidylcholine dissolved ether solution and buffer solution to evaporate ether to produce liposome.

And, double enteric microcapsule containing lectin is produced by coating the microcapsule mentioned above or liposome with alginic acid-salt which is stable in the stomach but decomposable in the intestine.

The following examples describe this invention in a detailed way. However, the following examples don't define the scope of the invention and it can be modified for the person who has ordinary skilled in this field of invention within the technology and claims of this invention.

Moreover, following examples describe the invention but it does not limit the boundary of the claim.

### Advantageous Effects

This invention is to produce an effective composition for enteric coated medicine of mistletoe extract containing lectin and producing an effective composition for enteric coated microcapsule which has lectin as a main ingredient. The problem with oral administration due to unstability in the digestive system was solved and it enhanced drug efficiency, medical cure effectiveness contributing to medical industry.

### BEST MODE FOR CARRYING OUT THE INVENTION

### Example 1

### Production of Lectin Containing Mistletoe Powder Extract, Water Extract, Powder from Water Extract

The lectin from various plants such as Cornus officinalis, an evening primrose, a kidney bean, beans, Liriope muscari, peony, sangrytk, papaya, a ground cherry, todangui, ivy, goosefoot, guallugun, shitake mushroom, Pinellia ternata, the boxwood tree, an acacia and also various natural substances from the sea such as starfish and mudfish can be produced similarly to mistletoe, however in this invention the mistletoe is used and it will be described below.

Classify mistletoe's sprout into leaf, fruit and stem and wash it with distilled water then lyopilize or dry in well ventilated place under 35° C. The powder was produced under 35° C. using roll crusher, ball mill and other mill machines or a grinder. Also, fresh mistletoe was freezes by liquid nitrogen in the same way. Size of the particle was controlled according to their use. It was confirmed that 1 mg of the powder contains 92 ng of lectin (VCA).

The extract was produced using the method (Korean Patent Application No. 2000-83383 and 2001-0061118) developed by this inventor. Wash each parts of leaf, fruit and stem and store them in −70° C. Add 10 times the amount of water than the material while grinding and mix it in 4° C. for 24 hours. Filter this through the gauze and centrifuge at 12,000 rpm for 30 minutes and filter the supernatant through the membrane filter from size 20 μm, 0.45 μm and 0.22 μm to remove the germs and add sterilized PBS buffer to adjust the concentration of the solution to 100 mg/mL. 100 mg/mL means in 1 mL solution there is 100 mg of mistletoe extract and when analyzing the quantity by ELLA (enzyme binding lectin detector), in 1 mg/ml concentrated VCE 1 mL, there was 30 ng of lectin (VCE). Also, filtered solution by filter membrane mentioned above was lyophilized into brown powder.

The Korean mistletoe (Viscum album L. var. coloratum) lectin (VCA) extract was produced from the method (Korean Patent Application No. 2000-83383 and 2001-0061118) mentioned above and purified it using asialofetuin-Sepharose, and the concentration was measured by BCA method and also lectin activity was measured by agglutination reactions of the blood cells.

### Example 2

### Granule Forming Process

### Example 2-1

The seed granule which is made in ratio of cellulose: starch: sugar: gelatin=30:30:30:10 was mixed with mistletoe extract or the lectin solution mentioned above with waring blender. The wet lump was dried in 4° C., vacuum, and grinded into pieces to form appropriate-sized particles and passed it through a sieve, stored it in 4° C., vacuum.

### Example 2-2

From example 1, the lectin as a power or water extracted powder 150 g, mannitol as a excipient 115 g, Avicel PH 101, 18 g, calcium-phosphate dibasic 17 g was mixed and floated in the fluid bed coater and sprayed the binding solution (propylmethylcellulose 20 g, water 100 mL and ethanol 100 mL) to it to produce seed granule for coating. The excipient used was mannitol, starch, lactose, and water or glucose and PV K-30, Avicel and water or lactose, mannitol, Ac-di-sol, hydroxypropylcellulose and 70% ethanol, or starch, lactose, sodium alginate and water or mannitol and white sugar mixed in appropriate rates. Mistletoe and the excipient inside the coater must stay in 25˜50° C. The incoming air and ventilation temperature was 35˜70° C. and the rotor was used in 100˜350 rpm.

### Example 3

### Enteric Coating Process

### Example 3-1

### Enteric Granule Coating Process

The granule from example 2 was used as seed and floated in the fluid bed coater and sprayed the coating solution (Zein-DP 25 g, shellac 35 g, 80% ethanol 180 mL) to coat. Inside the coater the temperature of the materials was controlled to be in 25˜60° C., the incoming air and ventilation temperature to be in 35˜70° C. and the rotor in 100˜350 rpm.

Coating reagent in this process are used singularly or in a complex using methacrylic acid copolymer Eudragit (registered a trademark) B-100, Eudragit L3D (Rohm & Hass company, Germany), corn protein extract (Zein-DP) and artificially processed goods using these to produce for instance, sodium alginate, alginic acid, shellac, carboxyvinylpolymer (carbomer (registered a trademark)), hydroxypropylmethylcellulose phthalate, hydroxypropylmethylcellulose acetate succinate, hydroxyproprylmethyl acetate succinate, carboxymethylcellulose, cellulose acetate phthalate, hydroxypropylcellulose, ethylcellulose, methylcellulose, polyvinylacetate phthalate, soy protein, wheat protein, processed goods using soy or wheat proteins, chitin, chitinic acid, processed goods using chitin or chitinic acids, gelatin, Carrageenan, pectin, Guar gum, Locust bean gum, Xanthan gum, Gellan gum, Arabic gum, Kollicoat MAE 30 DP (BASF company), medium chain triglycerides which has 6˜12 carbons. The coating reagent is desirable to have 1˜50 weight percent in the whole coated granule.

Producing primary coated granule using one of the composition listed above, double coating granule was produced using another coating solution.

### <Example 3-2> Enteric Tablet Coating Process

Plant dry powder or plant extract powder and lectin dried powder and excipient powder was passed through 48 mesh sieve to be in an appropriate ratio and magnesium stearate 5 mg for lubricant was added, mixed evenly and compressed into a tablet. For the excipient, various hydroxypropylmethylcellulose (HPMC) derivatives and EC, MC and others were used for dry, direct compression processing.

Coating reagent in this process are used singularly or in a complex using methacrylic acid copolymer Eudragit (registered a trademark) E-100, Eudragit L3D (Rohm & Hass company, Germany), corn protein extract (Zein-DP) and artificially processed goods using these to produce for instance, sodium alginate, alginic acid, shellac, carboxyvinylpolymer (carbomer (registered a trademark)), hydroxypropylmethylcellulose phthalate, hydroxypropylmethylcellulose acetate succinate, hydroxyproprylmethyl acetate succinate, carboxymethylcellulose, cellulose acetate phthalate, hydroxypropylcellulose, ethylcellulose, methylcellulose, polyvinylacetate phthalate, soy protein, wheat protein, processed goods using soy or wheat proteins, chitin, chitinic acid, processed goods using chitin or chitinic acids, gelatin, Carrageenan, pectin, Guar gum, Locust bean gum, Xanthan gum, Gellan gum, Arabic gum, Kollicoat MAE 30 DP (BASF company), medium chain triglycerides which has 6˜12 carbons. The coating reagent is desirable to have 1˜50 weight percentage in the whole coated tablet.

Producing primary coated granule using one of the composition listed above, double coating granule was produced using another coating solution.

### Example 4

### Lectin Containing Enteric Coated Microcapsules Process

### Example 4-1

### Microcapsulation Using Double-Emulsification Solvent Evaporation

1 N acetic acid solution containing lectin solution was poured into PLGA/CH₂Cl₂ 4mL and using the tissue crusher the primary emulsion solution was poured into 1% polyvinylalcohol 50 mL slowly to make secondary emulsion solvent. After mixing for three hours and removing CH₂Cl₂ it was centrifuged for 5 minutes in 2300 rpm and the supernatants were removed. Then it was washed with water 3 times to obtain microcapsule.

### Example 4-2

### Liposome Process

Appropriate concentration of lectin 3 mL was added to phosphatidyl choline (PC)/diethylether solution and homogenized the mixture with an ultrasonic generator until it became one phase. Nitrogen gas was passed through to remove diethylether completely and substances which were not enclosed in liposome were removed by Sephadex G-25 column. Centrifuged liposome precipitation was diluted with phosphate buffer 2 mL and Triton X-100 was added to destroy liposome. The capture efficiency of lectin measured was 68%.

### Example 4-3

### Alginic Acid Salt-Double Microcapsule Process

Surface active agent Span 80 2 mL and edible oil 48 mL were mixed and these were mixed with sodium alginate solution 8 mL and the microcapsule from example 4-1 or liposome from 4-2 diluted in 0.15 M NaCl solution. When the two layers were emulsified CaCl₂ solution (0.02˜0.2 M) 60 mL was poured smoothly and quickly until water/oil emulsion was upset and mixed this for 30 minutes leaving it as it was. When the alginic acid salt double microcapsule was formed, it was centrifuged and the supernatant was removed. The precipitated microcapsule was washed with water and acetone repeatedly and dried in room temperature. Dried alginate acid salt-double microcapsule 0.05 g was put into phosphate buffer (pH 7.4) 10 mL and mixed in 37° C. The capture efficiency of lectin after the filtration was 59%.

### Experimental Example 1

### Granularity Distribution of Mistletoe Enteric Coated Granule

Within the granule forming process from Example 2, lectin containing mistletoe powder or water extract powder 150 g, Avicel PH101 18 g and calcium-phosphate dibasic 17 g was mixed and binding solution (hydroxypropylmethylcellulose 20 g, water 100 mL and ethanol 100 mL) was sprayed to make the granule float as a seed in the fluid bed coater and coating solution 1 (Zein-DP 25 g, shellac 35 g, 80% ethanol 180 mL) was sprayed to measure the granularity distribution of coating granule 1. Also, the primarily coated granule was coated with coating solution 2 (Eudragit L30 165 mL, water 30 mL, acetic acid triethyl 5 g) to measure the granularity distribution of coating granule 2 and it appeared to be evenly distributed as 30˜40 mesh coated granules more than 80% (Table 1).

TABLE 1
The granularity distribution (%) of
mistletoe enteric coated mistletoe

| | Mesh size (mm) | | | |
|---|---|---|---|---|
| | 20(0.84) | 30(0.59) | 40(0.42) | 50(0.30) |
| Granularity distribution | 7.2 | 52.5 | 36.2 | 4.1 |
| of Example 1(%) | | | | |
| Granularity distribution | 15.2 | 60.3 | 20.6 | 3.9 |
| of Example 2(%) | | | | |

### Experimental Example 2

### Elution Rate of the Enteric Coated Granule and Tablet in Artificial Gastric Juice, Intestinal Juice

The elution rate of enteric coated granule 1 and 2 from Experimental Example 1 and mistletoe enteric coated tablet produced from Example 3-2 in artificial gastric and intestinal acids was measured. The samples were analyzed by preparing them every 15 minutes for an hour and later every 30 minutes using rotary specimens tube method under No. 1 dissolution test according to the 7^{th} revision of Korea Pharmacopeia. That is to say, putting the coated granules 1 and 2 in artificial gastric juice (pH 1.2, NaCl-HCl buffer solution) and artificial intestine juice (pH 6.8, phosphate buffer) 100 mL each and then in 37° C., mix it at 100 rpm to measure filtered lectin concentration by BCA method in regular time. Elution rate measured in water doesn't need mixing so the materials were left for certain time and was taken for measurement. The results are on FIG. 2. In the case of enteric coated granule 1 and 2, they didn't flow out in the artificial gastric juice but in the artificial intestine juice, they flowed out within 30 minutes. Also to get eluted 50% in the water, it took more than one week. Therefore, the coated granule can be applied to syrups, juices, beverages, milk, yogurt and others. In the case of enteric coated tablets, it took 5.5 hours to get 50% elution in artificial gastric acid and an hour in artificial intestine.

### Experimental Example 3

### The Surface Structure and Granularity Distribution of Alginic Acid Salt-Double Microcapsule

Measuring lectin containing alginic acid, salt-double microcapsule produced from Example 4-3 using Light scattering particle analysis, the average particle size was smaller as the concentrations for CaCl₂ and sodium alginate went smaller (Table 2).

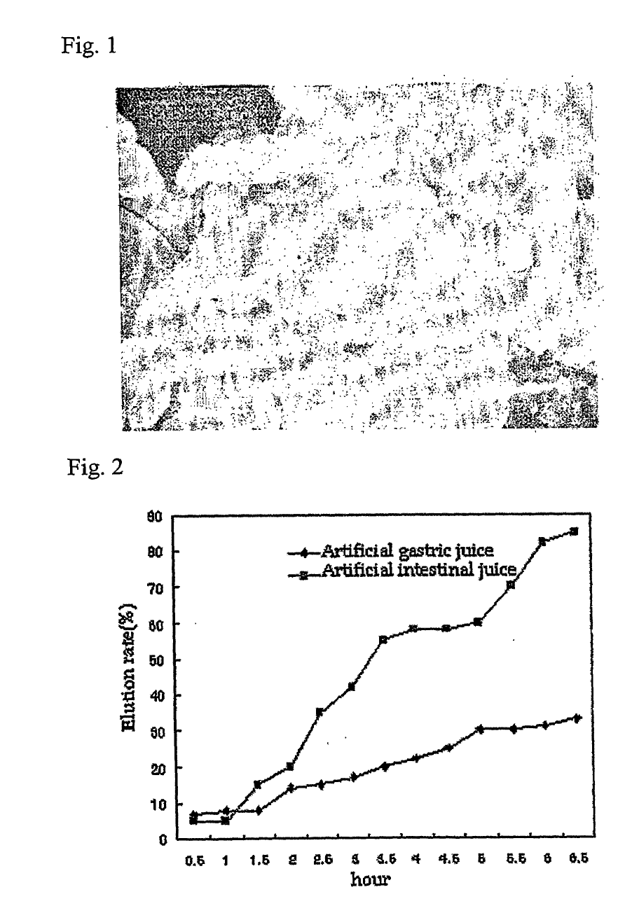

Sodium alginate double microcapsule which is made from 4% sodium alginate solution and 0.2 M CaCl₂ was completely dried at room temperature and was observed with scanning SEM, the microcapsule particle was in a spherical shape (FIG. 1).

TABLE 2
Particle size of the lectin containing
alginic acid salt-double microcapsule.

| CaCl₂(M) | Sodium alginate(%) | Particle size |
|---|---|---|
| 0.2 | 4 | 74.17 |
| 0.02 | 4 | 47.25 |
| 0.002 | 4 | 17.39 |

### Experimental Example 4

### Lectin Elution from Alginic Acid Salt-Double Microcapsule

The elution rate of lectin containing alginic acid salt-double microcapsule from example 4-3 was measured in artificial gastric juice and artificial intestine. Using rotary specimens tube method under No. 1 dissolution test according to the 7^{th} revision of Korea Pharmacopeia, the samples were analyzed every 30 minutes. Namely, the alginic acid salt double microcapsule 1 g was put into protease and lipase containing artificial gastric juice (pH 1.4, NaCl-HCl buffer solution) and intestinal juice (pH 7.4, phosphate buffer solution) 100 mL each and mixed in 37° C., 50 rpm. Next, filtered it in regular time and added Triton X-100 to destroy liposome and measured lectin by BCA method. The elution rate of lectin from alginic acid salt-double microcapsule in the artificial gastric juice after 6 hours was 31% and in the artificial intestine juice after 6 hours was 85%.

### Experimental Example 5

### In Vitro Anti Cancer Activity Towards Cancer Cells by Enteric Coated Medicine

The anti cancer activity of the eluted liquid of mistletoe enteric coated granule which has been treated for 6 hours in artificial intestine juice from Experimental Example 2 and the eluted liquid of alginic acid salt-double microcapsule which has been treated the same from Experimental Example 4 was analyzed by MTT analysis. Concentration of sample for coated granule was changed into mistletoe weight used in producing the granule and for alginic acid salt-double microcapsule was changed into amount of lectin used in producing liposome.

The cancer cell was plated in 96 well plate and various concentrations of samples were added and after 48 hours, the cell growth was examined using MTT method to show IC₅₀ (inhibitory concentration) which is a value for the sample concentration when killing 50% of the cancer cells.

According to the results, as Table 3 shows, in the case of enteric coated granule, the IC₅₀ value increases after the coating process which means it might had a little activity loss during the process or elution, however it showed anti cancer activity for oral cavity carcinoma, pharynx cancer, cervical cancer, stomach cancer, liver cancer, breast cancer, myelogenous leukemia and others which deserves our attention. Also, in the case of alginic acid salt-double microcapsule, the value increased similarly so it might have activity loss during the process or elution but it had remarkable anti cancer activity towards cancer cells.

TABLE 3
Cytotoxicity(IC₅₀) against various cancer cells by samples.

| | | Enteric coated granule | | | |
|---|---|---|---|---|---|
| | | (μg/mL) | | Alginic acid salt-double | |
| | | Before | After | microcapsule(μg/mL) | |
| Cancer cells | | coating(A) | coating(B) | Lectin(C) | Microcapsulation(D) |
| Skin cancer | B16-BL6 | 120 | 155 | 30 | 45 |
| Oral cavity | A253 | 5 | 7 | 3 | 7 |
| carcinoma | KB | 5 | 14 | 4 | 10 |
| Pharynx cancer | Fadu | 10 | 15 | 2 | 5 |
| Cervical cancer | SNU17 | 5 | 9 | 7 | 28 |
| | SNU778 | 23 | 29 | 8 | 15 |
| Bladder cancer | T24 | 150 | 198 | 10 | 18 |
| | J82 | 80 | 123 | 8 | 15 |
| Liver cancer | SK-Hep-1 | 120 | 156 | 8 | 12 |
| | Hep-3B | 14 | 23 | 5 | 18 |
| Stomach cancer | SNU-1 | 20 | 34 | 7 | 22 |
| Breast cancer | Hs 578T | 10 | 15 | 7 | 13 |
| Myelogenous | HL-60 | 7 | 12 | 3 | 10 |
| leukemia | | | | | |

A: Mistletoe powder weight(μg/mL) used in producing extract 1 mL.
B: Mistletoe powder weight(μg/mL) used in producing eluted liquid 1 mL from treating enteric coated granule in artificial intestine juice for 6 hours.
C: Concentration of mistletoe lectin(ng/mL).
D: Lectin weight(ng/mL) used in producing eluted liquid 1 mL from treating alginic acid salt double microcapsule made from lectin in artificial intestine juice for 6 hours.

1. A composition for an enteric coating of natural product containing lectin, which comprises:
mannitol 115 g as a excipient, Avicel PH 101 18 g, calcium-phosophate dibasic 17 g, hydroxypropylmethylcellulose 20 g as a binder solution, water 100 mL, ethanol 100 mL, Zein-DP 25 g as a coating solution, Shellac 35 g and 80% ethanol 180 mL.

2. The composition according to claim 1, wherein said excipient comprises mannitol, starch, lactose and water or glucose and PV K-30. Avicel and water or lactose, mannitol, Ac-di-sol, hydroxypropylcellulose and 70% ethanol, or starch, lactose, sodium alginate and water or mannitol and white sugar.

3. The composition according to claim 1, wherein coating solution are comprised of coating materials used singularly or in a complex using corn protein extract and its artificially processed goods, soy protein, wheat protein and its artificially processed goods and its derivatives, methacrylic acid copolymers, hydroxypropylmethylcellulose phthalate (HPMCP), hydroxypropylmethylcellulose acetatesuccinate (HPMCAS), cellulose acetate phthalate, polyvinylacetate phthalate, alginic acid salts, shellac, carboxyvinylpolymers, carboxymethylcellulose, hydroxypropylcellulose, ethylcellulose, methylcellulose, chitin, chitinic acid, processed goods using chitin or chitinic acids, gelatin, Carrageenan, pectin, Guar gum, Locust bean gum, Xanthan gum, Gellan gum, Arabic gum, Kollicoat MAE 30 DP, medium chain triglycerides which has 6˜12 carbons.

4. The composition according to claim 1, wherein natural product containing lectin is mistletoe.

5. A composition for an enteric coated microcapsules containing lectin, which comprises:
PLGA/CH₂Cl₂ 4 mL, 1% polyvinylalcohol 50 mL, Span80 2 mL as a surface active agent, edible oil 48 mL, 1˜4% sodium alginate solution 8 mL and 0.02˜0.2 M CaCl₂ solution 60 mL.
